(19) United States
(12) Patent Application Publication
(10) Pub. No.: US 2015/0010636 A1
(43) Pub. Date: Jan. 8, 2015

(54) Apparatus and Method for Reducing the Occurrence of Post-Surgical Adhesions

(71) Applicant: John P. Delaney, Minneapolis, MN (US)

(72) Inventor: John P. Delaney, Minneapolis, MN (US)

(73) Assignee: ANHESE LLC, Minneapolis, MN (US)

(21) Appl. No.: 14/482,187

(22) Filed: Sep. 10, 2014

Related U.S. Application Data
(63) Continuation of application No. 13/302,563, filed on Nov. 22, 2011.
Application No. 13/302,563 is a continuation-in-part of application No. 12/773,480, filed on May 4, 2010, now Pat. No. 8,227,084.

(51) Int. Cl. A61K 31/436; A61K 31/341; A61M 5/145; A61K 47/10; A61K 9/00; A61K 31/16; A61K 47/24

(52) U.S. Cl. CPC A61K 31/436; A61K 31/16; A61K 31/341; A61K 47/24; A61K 47/10; A61K 9/0012; A61M 5/145; A61M 2005/14513; A61M 2205/04
USPC 424/489; 514/625; 514/473; 514/291; 604/506

(57) ABSTRACT

A method for inhibiting formation of adhesions following abdominal surgery which involves application of an anti-static fatty acid ethoxylated amide (Cocamide DEA) in a matrix that is placed in the peritoneal cavity at the conclusion of an abdominal surgery and which releases this anti-adhesive chemical over a predetermined time in a range up to seven days. Tests conducted on laboratory rats established that the method reduced the incidence of adhesions from 100 percent (100%) in a test model to near zero in the majority of treated animals. In an alternative embodiment, andrographalide was delivered via a pump with similar results. In still another embodiment, an effective amount of 50% phosphatidylchorene and propylene glycol was delivered, via a pump, into the abdominal cavity, again with similar results.

### CROSS-REFERENCE TO RELATED APPLICATIONS

The present application is a continuation of application Ser. No. 13/302,563, filed Nov. 22, 2011 which is a continuation-in-part of application Ser. No. 12/173480, filed Jul. 15, 2008, which claims priority to a provisional application serial no. 60/956,560, filed Aug. 17, 2007.

### BACKGROUND OF THE INVENTION

1. Field of the Invention

This invention relates generally to the field of abdominal surgery, and more particularly to methods for reducing or substantially eliminating formation of adhesions following such abdominal surgery.

2. Discussion of the Prior Art

Abdominal adhesions are bands of fibrous tissue that cause abdominal organs to adhere to one another or to the abdominal wall. Common examples are intestine-to-intestine, and intestine-to-pelvic organs, intestine-to-abdominal wall and omentum to any of these sites. Adhesions can develop as after-effects of peritonitis, or of abdominal trauma. However, in developed countries, such adhesions most commonly result from abdominal surgical procedures during which organs are traumatized by surgical manipulations.

In most patients, post-surgical adhesions do not produce adverse consequences. In some individuals, however, constricting adhesions block the flow of contents through the intestines, a condition called intestinal obstruction. In certain instances, a segment of bowel becomes twisted around an adhesive band, thus cutting off the normal blood supply, The affected portion of the intestine becomes non-viable and may perforate. This requires emergency surgery for corrective action. In the U.S., each year about 100,000 operations are carried out to alleviate intestinal obstructions,

Once abdominal adhesions have formed, they do not resolve. Their lysis, by operation, only temporarily eliminates them. For example, when surgery is performed for adhesive intestinal obstruction, adhesions routinely re-form and later cause a new intestinal obstruction in 11%-21% of such cases.

Another complication of adhesions is female infertility, secondary to blockage of fallopian tubes. Surgical attempts to solve this problem often fail because of reformation of adhesions. Furthermore, many gynecologists are of the opinion that pelvic adhesions cause pelvic pain and they operate to divide them with the intent of alleviating the pain.

Minimally invasive techniques for abdominal procedures reduce tissue handling, but even with this approach, post-operative adhesions occur in most patients. One strategy to minimize post-surgical adhesion formation might be combining minimally invasive techniques with anti-adhesion agents. At the present time, the majority of abdominal operations are still done through a large abdominal wall incision with direct exposure.

Numerous agents have been employed clinically with the hope of preventing postoperative adhesions. A few have proved partially successful in that they reduce, but do not eliminate, subsequent adhesion formation. Available products all are site specific and are not intended to solve the problem throughout the abdomen. This is a limited benefit because the locations of future adhesions are not entirely predictable.

Some of the currently used products include hyaluronic acid and/or carboxy methylcellulose. Some are fabricated as a film, others in a sponge-like configuration. They must be applied in a selected fashion directly to the surfaces of the specific organs or areas where adhesions might be expected to form or where they would be particularly troublesome, such as over the pelvic organs. Others are constituted are viscous gels which are painted on or sprayed on specific injured sites.

Examples of commercial products are as follows:

Alliance Pharmaceutical Corporation markets a product sold under the trademark, FloGel®, which is a thermal-reversible gel comprising biocompatible polyoxamers made up of polyoxyethylene and polyoxypropylene units, it has the ability to change from a liquid to a gel upon warming to body temperature. Applied in the liquid state, it will mold to tissue contours before gelling in place. Thus, FloGel maintains contact with tissue surfaces and serves as a physical protective barrier to adhesion formation.

Absorbable fabric barriers have been developed to prevent post-surgical adhesions. A product of the Ethicon subsidiary of the Johnson & Johnson Corporation has developed a barrier fabric which it advertises under the trademark, Gynecare Interceed®, to be placed during gynecologic surgery to reduce the incidence of pelvic adhesions.

Genzyme Corporation markets a product called SEPRAFILM® which provides a temporary physical barrier for separating potentially adhesiogenic tissue surfaces during the critical five to seven day period when adhesions generally form. The material is bio-resorbable.

There is currently an array of similar products, none of which really eliminates adhesions and all of which are site specific. Mechanical barrier agents, generally in the form of films and sponges, are the most commonly employed and most practical agents in clinical use today for preventing adhesions in the abdominal cavity. They serve to separate raw or injured areas and, hence, mechanically block adhesions from forming, but only at selected sites. Absorption occurs by enzymatic degradation and physiological uptake. The residence time in the abdomen varies according to the agent involved. The physical integrity of the film or sponge may dissipate within a day or two after operation. The continued prevention of adhesions following the disappearance of the materials may be due to residual biochemical effects of the harrier agents. Materials used in films have included polyglycolic acid, polylactic acid, oxidized cellulose, hydrophilic polyethylene glycol and sodium hyaluronate with carboxymethylcellulous. The latter agent is sold under the trademark, SEPRAFILM®. Of the various agents proposed, the bioresorbable barrier agent, SEPRAFILM, has yield the most significant impact in the clinical setting.

### SUMMARY OF THE INVENTION

In order to minimize or eliminate intra-abdominal adhesions following an operation, we have discovered that by introducing a polymeric matrix impregnated with an anti-static agent, Cocarnide DEA, into the abdominal cavity remote from the predictable adhesion sites at the time of surgery and left in place for a period of time in a range of from three to seven days, subsequent adhesion formation is inhibited and most often eliminated. It should be noted that there exist numerous identical or nearly identical anti-static chemicals. The matrix may comprise, but is not limited to, polyethylene, formed as a sheet, which may be passed through the abdominal wall in the same fashion as a conventional surgical drain. The polymer matrix, preferably polyethylene, slowly releases the amide antistat into the peritoneal fluid at a rate that has empirically proved to be sufficient to inhibit adhesion formation. After the five to seven days, the plastic sheet is pulled out like a drain.

Our experiments have also established that the drugs including Rapamune, andrographolide and Phosal® 50, when delivered from an implanted mini osmotic pump over a period starting at the operation and continuing for about three to seven days, post surgery, also significantly reduces the formation of surgical adhesions in lab animals.

### DISCUSSION OF THE PREFERRED EMBODIMENT

In carrying out the present invention, a polymeric substrate, which has been impregnated with an ethoxylated fatty acid amide, such as Cocamide DEA, is left to reside in the abdominal cavity following surgery for a predetermined length of time. Without limitation, a sheet of the anti-static polymeric material may be rolled to form a tube-like configuration to facilitate passage through a surgically created opening in the abdominal wall, using a trocar or similar instrument as used in laparoscopic surgery. A percutaneous tether can be affixed to the proximal end of the coiled substrate, in contrast to other products, the amide impregnated substrate need not be in physical contact with the injured sites to inhibit formation of adhesions. Over time, the Cocarnide DEA diffuses from the elastomeric substrate and mixes with abdominal fluids thereby effectively bathing all peritoneal surfaces. Without limitation, the substrate may preferably be fabricated from a sheet of polyethylene. As later explained, other materials and vehicles may be substituted as a carrier matrix for the drug without departing from the scope of the invention. Specifically, polyester or polyethylene sheets treated with the anti-static chemical can be employed. As an alternative, bioabsorbable microspheres currently used for drug delivery can be used as a slow-release media, as can gels, micelles, or liposomes. Polymeric micelles are nano-sized particles that are made up of polymer chains and are usually spontaneously formed by self-assembly in a liquid, generally as a result of hydrophobic or ion pair interactions between polymer segments. They typically have a so-called “core-shell” structure. The core of the micelles, which is either the hydrophobic part or the ionic part of the nano particles, can contain small molecules such as therapeutic drugs, while the shell provides interactions with the solvent to make the nano particles more stable in a liquid. Additionally, other anti-adhesive agents, Rapamune, Phosal® 50 (50% phosphatidylcholine in propylene glycol) or andrographolide might be delivered by these same means for the same purpose. Alternatively any of these agents be infused by a pump.

### EXPERIMENTAL BACKGROUND

The efficacy of the slow release of Cocamide DEA into the abdominal cavity in preventing adhesion formation has been confirmed in a series of experimental studies in which Sprague-Dawley female rats were used. Two models were devised that both induced adhesions in 100 percent of experimental animals.

The first is termed mesh model (M). A patch of polypropylene surgical mesh, approximately 2.5 cm square is sewn into a surgically created defect in the mid-abdominal wall of a Sprague-Dawley rat.

The time, course and extent of adhesions to the mesh was determined by repeated laparoscopic observation at predetermined intervals on days 1, 3, 5, 7, 14, 28 and 150 following mesh placement. It was found that in untreated control animals, extensive adhesions were seen at day one and then progressively covered larger areas of the mesh surface for up to seven days, after which no further adhesions developed.

Another series of Sprague-Dawley female rats was subjected to the same surgical procedures, but a polyethylene plastic film impregnated with the anti-static lipid amide, 7 cm by 7 cm in size, was left in the abdomen at the end of the operation, fixed in a position remote from the surgical mesh.

One hundred percent of the control animals developed adhesions to the prosthetic mesh. The average area covered was over 90 percent. The smallest area of coverage was 70 percent. When the anti-static impregnated plastic was placed, most of the rats developed no adhesions. In a few instances, small adhesions developed at the junction of the mesh to the abdominal wall, but did not involve the surface area of the mesh.

The second model included no foreign materials and is designated tissue model (T). This involved an identical excision of a segment of abdominal wall with simple skin closure, In this mariner, the abdominal viscera were exposed to the subcutaneous tissue. The significance is that this represents a different stimulus for the adhesion process to occur involving only native tissue and no foreign material.

Using this tissues model, we observed the same degree of adhesion inhibition as was previously found in the mesh model, when the anti-static polyethylene film was present.

Based on these experiments, we concluded that the presence of the plastic, impregnated with this fatty acid amide, left within the peritoneal space inhibited adhesions to the adhesiogenic surface, M or T, very probably by the slow release of the drug from the film matrix into and throughout the abdominal cavity.

To determine further if the anti-static plastic providing a slow release of Cocamide DEA inhibits adhesions in general, we carried out a similar study with yet a different model of adhesion induction. This preparation is called the “sidewall model” and is the most commonly employed experimental method for studying abdominal adhesions. A small segment of peritoneum was excised and the cecum was abraded. The presence of anti-static plastic led to much reduced adhesion formation as compared to controls, again confirming that this approach is widely applicable.

Additional studies demonstrated that untreated plain polyethylene film did not, by itself, alter the adhesion pattern, nor were adhesions inhibited when a dose of the anti-static agent in the liquid form was simply left in the abdomen at the time of mesh placement. This latter observation indicates that prolonged exposure to the chemical agent is needed to attain the desired effects.

The present invention offers unique approaches: (1) slow release of an anti-adhesive agent, such as Cocarnide DEA, which circulates through the abdominal cavity, and provides generalized and prolonged protection from adhesion formation; (2) a strategy which allows removing the foreign plastic substrate after the critical time interval needed for adhesion formation; and (3) the use of a class of amides that have been widely employed as anti-static agents in polymer packaging material. There are fairly extensive data in this context indicating low potential for toxicity. Such amides also have antioxidant and surfactant properties which may or may not play a role in adhesion prevention. It should be further noted that this category of chemicals is employed extensively in dermatological preparations. Cocamide is also used as an anti-static agent in polyethylene film intended for the packaging of electronic components to protect against build-up of static charges. The film used in our studies was embedded with the anti-static agent Alkamide Cdd 518 (Rhodia Novecare) during the manufacturing process. This compound is a long chain aliphatic acid with diethylamide groups. The molecules align themselves with the hydrophilic amide end extending to the surface of the film and binding water molecules. The combination is electrically conductive and, therefore, anti-static. In a liquid medium, the molecules diffuse out of the surface of the polyethylene film. The experiments we have outlined herein employed small squares of such material placed in the rat's abdomen. At this time, we do not fully understand how small concentrations of the chemical, slowly released into the peritoneal cavity of rats, following operations, act to inhibit the deposit of fibrin matrices between adjacent injured tissue structures, the precursors of adhesions.

It is of interest to note that the anti-static films removed after seven days in the rat abdomen, resterilized and used in a fresh M model rat yielded results identical to controls, that is, no effect on adhesions. Five such “used” films were submitted to the manufacturer where electrical assessments were made, namely, electrostatic decay rate (charge dissipation) and electrical resistance. These measurements indicated no residual anti-static properties, The anti-stat agent was fully dissipated over these seven days that the film remained in the rat's abdomen.

Next, we studied the results in the two models, infusing over a seven day period post surgery, a comparable dose of Cocamide DEA by means of a miniature pump left in the abdominal cavity. The results were virtually identical to those obtained with the anti-static polyethylene film, that is, near total elimination of adhesions in both models, M and T. It should be noted that the two methods each involved the presence of foreign material in the abdomen, polyethylene film or the cellulose casing of the pump. There seems to be a synergistic effect.

Another unrelated chemical, andrographolide, was tested for adhesion inhibition in the two models. Delivered by means of the intra-abdominal miniature pump over the course of a week after operation, this yielded adhesion reduction comparable to the anti-static film and to the infused alkamide.

Andrographolide is an irreversible blocker of NF-kB derived from a medicinal plant andrographis panniculata. It has wide ranging pharmacologic action including anti-oxidant and anti-inflammatory capabilities. Most pertinent to present consideration is its anti-coagulant capability, based on inhibition of tissue factor and thereby of the alternate pathway for extravascular coagulation, an essential first step for adhesion formation.

A third agent has proved to generate similar anti-adhesive effects in the same two rat models. Rapamune is a complex mixture prepared for oral administration to prevent rejection of transplanted organs or tissues in the human. The mixture includes Rapamycin, Phosal® 50 PG, polysorbate 80 and propylene glycol. We observed that this preparation infused into the abdomen for seven days following operation serves as a powerful adhesion inhibitor in the models M and T. Rapamycin, by itself, was found not to have this effect. We also explored the possible synergistic effect of using a tube of the anti-static film as an avenue for infusing the three agents individually and in various combinations. This strategy permitted a reduced amount of the plastic film in the abdomen. However, the quantity of film needed for the full effect in the rat, if translated, weight for weight to the human is deemed not to be practical.

Our continued work has led us to conclude that it is actually the Phosal® 50 ingredient of Rapamune that has the desired effect of suppressing a post-surgical adhesion formation. Phosal 50 is a mixture of 50% phosphatidyicholine with propylene glycol with trace amounts of sunflower mono-diglycerides and ascorbyl palmitate. The prolonged delivery of Phosal 50 from an external pump over a period of seven days was found to greatly reduce adhesions to implanted polypropylene mesh. Pure phosphatidylcholine, however, was found to be ineffective.

While not fully understood, the effects of anti-static polyethylene film might be exerted as an electrical phenomenon, possibly by attracting and inactivating platelets and thus the initial fibrin clotting process.

Another device now under investigation for drug delivery comprises bundles of hollow semi-permeable cellulose based microtubules of the type employed in blood oxygenators or in renal dialysis apparatuses. The anti adhesion agent is delivered from an external pump through the fibers or, alternatively, is loaded into the lumens of the fibers from which it slowly diffuses through the micro tubule walls from the static column.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A composition suitable for local administration of a drug into the peritoneal cavity following a surgical procedure, said composition comprising a 50% phosphatidylcholine plus propylene glycol in an amount effective to inhibit formation of post-operative adhesions upon intraperitoneal administration of said composition to tissues, throughout the peritoneal cavity in a carrier suitable for local prolonged administration of said composition with consequent minimal levels attained in the systemic circulation.

2. The composition of claim 1 wherein said carrier is selected from a group consisting of microspheres, nanospheres, fibers, polymeric films, gels , micelles and drug delivery pumps.

3. A method of inhibiting formation of abdominal adhesions, post-surgery, which comprises delivering an effective amount of the composition of claim 1 into the abdominal cavity at the time of the surgical procedure and the delivery continuing over a predetermined time interval.

4. The method of claim 3 wherein the predetermined time interval is at least three days.

5. The method of claim 3 wherein the composition is delivered into the abdominal cavity by an external drug infusion pump via one of a single catheter and a bundle of semipermeable microtubules.

6. The method of claim 3 in which the composition is delivered continuously from an implanted drug delivery pump and the predetermined time interval is no less than three days.

7. The method of claim 6 wherein the predetermined time period is at least seven days.

8-13. (canceled)

14. A composition suitable for local administration of a drug topically to tissue within the peritoneal cavity having been subjected to a surgical procedure, said composition comprising a cocamide DEA agent in an amount effective to inhibit formation of post-operative adhesions upon. local, non-systemic administration of said anti-adhesive agent to intra-abdominal tissues, and a carrier suitable for local prolonged administration of said anti-adhesive agent with minimal systemic circulation,

15. The composition of claim 14 wherein said carrier is selected from a group consisting of microspheres, nanospheres, hollow fibers, polymeric films, gels, micelles and drug delivery pumps.

16. A method of inhibiting formation of abdominal adhesions, post-surgery, which comprises delivering an effective amount of the composition of claim 14 into the abdominal cavity at the time of surgery and continuously over a predetermined time interval, following surgery of no less than three days and preferably at least seven days.

17. The method of claim 16 wherein the composition of claim 14 is delivered from an implanted drug delivery pump.

18. A method for inhibiting formation of adhesions following abdominal surgery comprising the step of applying a composition of an andrographolide agent in a local, non-systemic manner to intra-abdominal tissues by way of a carrier providing prolonged delivery of the andrographolide agent over a predetermined time period.

19. The method of claim 18 wherein the carrier is selected from a group consisting of microspheres, nanospheres, fibers, polymeric filons, gels, micelles and drug delivery pumps.

20. The method of claim 18 wherein the andrographolide is delivered into the abdominal cavity by a drug infusion pump through one of a single catheter and a bundle of semipermeable microtubules.

21. The method of claim 18 wherein the andrographolide agent is delivered by way of an implanted drug delivery pump and the predetermined time period is at least three days.

22. The method of claim 18 wherein the predetermined time interval is the at least seven days.
